(19) United States
(12) Patent Application Publication
(10) Pub. No.: US 2019/0038138 A1
(43) Pub. Date: Feb. 7, 2019

(54) ACOUSTIC WAVE APPARATUS

(71) Applicant: CANON KABUSHIKI KAISHA, Tokyo (JP)

(72) Inventors: Takashi Yoshida, Tokyo (JP); Takaaki Nakabayashi, Kawasaki-shi (JP)

(21) Appl. No.: 16/047,378

(22) Filed: Jul. 27, 2018

(30) Foreign Application Priority Data
Aug. 3, 2017 (JP) 2017-150346

(51) Int. Cl. A61B 5/00

(52) U.S. Cl. CPC A61B 5/0095

(57) ABSTRACT

An acoustic wave apparatus comprising a supporting base provided with an insertion opening into which an object is to be inserted; a liquid vessel arranged below the insertion opening configured to store acoustic matching liquid; and a receiving portion configured to receive an acoustic wave generated at the object and propagated via the acoustic matching liquid from; a bank portion protruding from the liquid vessel toward the supporting base, wherein the liquid vessel includes a bank portion at upper side configured to reduce an amount of the matching liquid over flowed from the liquid vessel due to a changing of a wavefront of the matching liquid, in a part of a circumferential direction of the liquid vessel, the bank portion is provided with a communicating portion through which the acoustic matching liquid can pass.

### BACKGROUND OF THE INVENTION

### Field of the Invention

The present invention relates to an acoustic wave apparatus.

### Description of the Related Art

Conventionally, photoacoustic imaging using a photoacoustic wave generated from an object irradiated with light and ultrasonic imaging using a reflected wave from an object to which an acoustic wave (for example, an ultrasonic wave) is transmitted are being studied and utilized in the field of medicine and the like. As a type of an acoustic wave apparatus which acquires object information using an acoustic wave in this manner, a known object information acquiring apparatus is provided with a supporting base which supports an object, a liquid vessel which stores an acoustic matching liquid, and a probe which receives an acoustic wave from the object via the acoustic matching liquid.

Japanese Patent Application Laid-open No. 2015-205041 describes an acoustic wave apparatus (a photoacoustic wave measuring apparatus) which receives a photoacoustic wave generated from an object such as a breast and obtains a photoacoustic image of the object. The acoustic wave apparatus according to Japanese Patent Application Laid-open No. 2015-205041 is provided with: a supporting base which supports an examinee in a prone position and which includes an insertion opening for a breast of the examinee; and a liquid vessel which is arranged below the supporting base, which supports a probe, and which stores an acoustic matching liquid. Japanese Patent Application Laid-open No. 2015-205041 discloses keeping a level of the acoustic matching material at a favorable position by connecting the supporting base and the liquid vessel with a seal member.

Patent Literature 1: Japanese Patent Application Laid-open No. 2015-205041

### SUMMARY OF THE INVENTION

In an acoustic wave apparatus provided with a supporting base and a liquid vessel as described above, there is a risk that a leakage may occur due to an overflow of the acoustic matching liquid in the liquid vessel and the object may get wet.

The present invention has been made in consideration of the above, and an object thereof is to provide, in an acoustic wave apparatus using an acoustic matching liquid, a technique for reducing wetting of an object due to leakage of the acoustic matching liquid.

The present invention provides an acoustic wave apparatus, comprising:

a supporting base provided with an insertion opening into which an object is to be inserted;

a liquid vessel arranged below the insertion opening configured to store acoustic matching liquid; and

a receiving portion configured to receive an acoustic wave generated at the object and propagated via the acoustic matching liquid from;

a bank portion protruding from the liquid vessel toward the supporting base, wherein

the liquid vessel includes a bank portion at upper side configured to reduce an amount of the matching liquid over flowed from the liquid vessel due to a changing of a wavefront of the matching liquid,

in a part of a circumferential direction of the liquid vessel, the bank portion is provided with a communicating portion through which the acoustic matching liquid can pass.

According to the present invention, in an acoustic wave apparatus using an acoustic matching liquid, a technique for reducing wetting of an object due to leakage of the acoustic matching liquid can be provided.

Further features of the present invention will become apparent from the following description of exemplary embodiments with reference to the attached drawings.

### BRIEF DESCRIPTION OF THE DRAWINGS

FIG. 1 is a sectional view of an acoustic wave apparatus according to a first embodiment;

FIGS. 2A and 2B are enlarged views of a bank portion of the acoustic wave apparatus according to the first embodiment;

FIGS. 3A to 3D are enlarged sectional views of the bank portion of the acoustic wave apparatus according to the first embodiment;

FIG. 4 is a plan view of a coupled receiving unit of an acoustic wave apparatus according to a second embodiment;

FIG. 5 is an enlarged view of a bank portion of the acoustic wave apparatus according to the second embodiment; and

FIG. 6 is a sectional view of a coupled receiving unit and a supporting base of an acoustic wave apparatus according to a third embodiment.

### DESCRIPTION OF THE EMBODIMENTS

Hereinafter, preferred embodiments of the present invention will be described with reference to the drawings. However, it is to be understood that dimensions, materials, shapes, relative arrangements, and the like of components described below are intended to be changed as deemed appropriate in accordance with configurations and various conditions of apparatuses to which the present invention is to be applied. Therefore, the scope of the present invention is not intended to be limited to the description given below.

The present invention relates to a technique for detecting an acoustic wave propagating from an object and generating and acquiring characteristic information (object information) of the inside of the object. Accordingly, the present invention can be considered an object information processing apparatus or a control method thereof, or an acoustic wave apparatus or a control method thereof. The present invention can also be considered an object information processing method or a signal processing method. The present invention can also be considered a program that causes an information processing apparatus including hardware resources such as a CPU and a memory to execute these methods or a computer-readable non-transitory storage medium storing the program.

The present invention includes a photoacoustic apparatus utilizing a photoacoustic effect in which an acoustic wave generated inside an object by irradiating the object with light (an electromagnetic wave) is received and characteristic information of the object is acquired as image data. In this case, characteristic information refers to information on a characteristic value corresponding to each of a plurality of positions inside the object which is generated using a signal derived from a received photoacoustic wave.

In addition, the present invention includes an apparatus utilizing an echo technique which irradiates an object with an acoustic wave and receives an acoustic wave (a reflected wave) reflected and scattered at a specific position inside the object and propagating from the object. Since such an object information acquiring apparatus obtains characteristic information of the inside of an object based on reflection and scattering characteristics of an acoustic wave in the form of image data and the like, the object information acquiring apparatus can also be called an echo imaging apparatus. Characteristic information in an echo imaging apparatus refers to a difference in acoustic impedance inside an object, a position where a difference in acoustic impedance exists, sound velocity, and density.

Image data according to the present invention is a concept encompassing all image data derived from a photoacoustic wave or a reflected wave. For example, image data represents at least one piece of characteristic information such as initial sound pressure, energy absorption density, or an absorption coefficient of a photoacoustic wave, and a concentration of a substance constituting an object. Image data indicating a concentration of a substance constituting the object is obtained based on a photoacoustic wave generated by irradiating light with a plurality of wavelengths that differ from each other. Examples of data indicating a concentration include oxygen saturation, a value obtained by weighting oxygen saturation with intensity of an absorption coefficient or the like, total hemoglobin concentration, oxyhemoglobin concentration, and deoxyhemoglobin concentration. In addition, image data may indicate a glucose concentration, a collagen concentration, a melanin concentration, or a volume fraction of fat or water.

A two-dimensional or three-dimensional characteristic information distribution is obtained based on characteristic information at each position in the object. Distribution data may be generated as image data. While the generation of three-dimensional volume data by image reconstruction will be described as an example in the present invention, the present invention is not limited thereto. Characteristic information may be obtained as distribution information of respective positions inside the object instead of as numerical data. In other words, an initial sound pressure distribution, an energy absorption density distribution, an absorption coefficient distribution, or an oxygen saturation distribution may be obtained.

An acoustic wave according to the present invention is typically an ultrasonic wave and includes an elastic wave which is also referred to as a sonic wave or an acoustic wave. A signal (for example, an electrical signal) transformed from an acoustic wave by a transducer or the like is also referred to as an acoustic signal or a received signal. However, descriptions of an ultrasonic wave and an acoustic wave in the present specification are not intended to limit a wavelength of the elastic waves.

### First Embodiment

### Apparatus Configuration

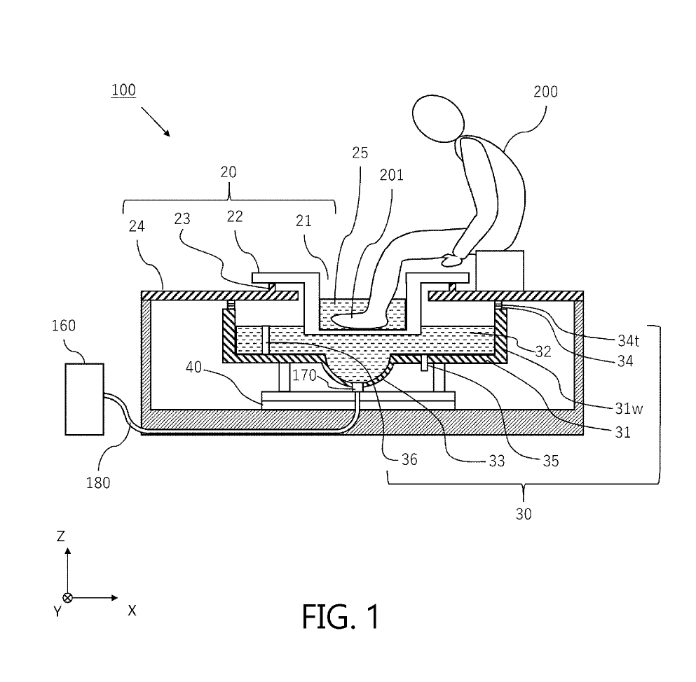

FIG. 1 is a sectional view showing an acoustic wave apparatus 100 according to a first embodiment. The acoustic wave apparatus 100 is roughly provided with a supporting base 20, a coupled receiving unit 30, and a scanning unit 40.

Supporting Base

The supporting base 20 is a base having a supporting surface that supports an examinee 200. In FIG. 1, a supporting top plate 24 with sufficient strength is provided as the supporting surface. An insertion opening 21 into which an object 201 that is a part of the examinee 200 is to be inserted is provided on the supporting surface. In FIG. 1, a foot is used as the object 201. As another example, a breast of the examinee 200 in a prone position on the supporting base 20 may be used as the object 201.

A holding member 22 for holding the object 201 is provided in the insertion opening 21. An object-side acoustic matching liquid 25 for matching acoustic impedances of the holding member itself and the object 201 may be stored inside the holding member 22. Water, oil, or the like is favorable as the object-side acoustic matching liquid 25. The supporting top plate 24 is provided with an securing portion 23 for the purpose of securing the holding member 22. The securing portion 23 supports the holding member 22, and a support method thereof may be a watertight structure support by an O-ring or the like or a discrete contact support such as support at a plurality of points by a load cell.

In other words, the supporting top plate 24 is configured so that the holding member 22 holding the object can be connected at a position overlapping the insertion opening 21.

At least a part of the holding member 22 is constituted by a member capable of transmitting an acoustic wave propagated from the object. For example, PET or acrylic can be used. In addition, the holding member 22 may be configured by providing a sheet member capable of acoustic transmission such as a film on a lower surface of a cylindrical member. In doing so, a reinforcing member such as a meshed wire may be used to reinforce the film. Furthermore, when the object 201 is a breast, a cup-shaped member that conforms to the shape of the breast may be used as the holding member 22. Since the holding member 22 separates the object-side acoustic matching liquid 25 from an acoustic matching liquid 32 stored in a liquid vessel 31, the holding member 22 can also be called a liquid separating portion.

Coupled Receiving Unit

The liquid vessel 31 capable of storing the acoustic matching liquid 32 is provided in the coupled receiving unit 30. The coupled receiving unit 30 including the liquid vessel 31 is arranged below the insertion opening of the supporting base 20.

The liquid vessel 31 includes a bottom portion and a liquid vessel wall 31w, and forms a container configured to store and holding the acoustic matching liquid 32. Although details will be given later, the liquid vessel 31 is provided with a bank portion 34 as a characteristic component. The liquid vessel 31 is strong enough to withstand water pressure when full or forces applied during scanning and favorably constituted by metal, a resin, or the like. The liquid vessel 31 may have any shape when viewed from above. A circle, an ellipse, a rectangle, a rectangle with rounded corners, or the like may be determined in accordance with conditions including a type of the object and configurations of other members of the apparatus.

A supply port 35 is provided in a part of the liquid vessel 31 configured to supply the acoustic matching liquid 32. The supply port 35 may double as a recovery port 37 for performing recovery, or the recovery port 37 may be separately provided. The supply port 35 and the recovery port 37 are connected by a tube or the like to a supply unit (not shown) provided with a pump, a tank, a sterilizing mechanism, a supply control unit, and the like. The supply unit may be provided inside the acoustic wave apparatus 100 or may be provided outside the apparatus and connected to the apparatus by a tube. The supply control unit controls the pump and the like based on sensed data of a liquid level detecting unit 36 to be described later, instructions from a user via input means, and the like, and controls a liquid level of the acoustic matching liquid 32. The supply control unit may be constituted by an independent control circuit or an information processing unit (to be described later) may double as the supply control unit.

An receiving portion 33 which receives an acoustic wave generated from the object and converts the acoustic wave into an analog electrical signal is provided in a part of the liquid vessel 31. The receiving portion corresponds to the coupled receiving unit according to the present invention. Although the present diagram shows the receiving portion 33 being fixed to the bottom portion of the liquid vessel 31, the receiving portion 33 capable of moving independently of the liquid vessel 31 may be arranged inside the liquid vessel.

Any conversion element such as a piezoelectric element or a capacitive micro-machined ultrasonic transducer (CMUT) can be used as the receiving portion 33. In addition, configuring the receiving portion 33 by arranging a plurality of conversion elements in a linear pattern or an array enables a reduction in noise or a reduction in measurement time to be realized. As shown in FIG. 1, the receiving portion 33 is favorably constructed by arranging a plurality of conversion elements on a hemispherical supporting portion. Accordingly, a high sensitivity region is formed in which directions of high receiving sensitivity of the respective conversion elements concentrate, and imaging of the object can be performed with accuracy. An analog electrical signal is subjected to signal processing by a signal processing unit (not shown) including an AD converter and an amplifier, and then transmitted to a storage unit and the information processing unit to be described later.

The liquid vessel 31 is provided with the liquid level detecting unit 36 in order to detect a boundary position of the acoustic matching liquid 32. The liquid level detecting unit 36 is, for example, an optical sensor, a floating sensor, a capacitance sensor, of the like and is for checking that the acoustic matching liquid 32 is being supplied to a desired position.

Scanning Unit

The scanning unit 40 is a unit for driving the coupled receiving unit 30 in at least one direction and is constituted by a driving unit, a mounting, a controller, and the like. The coupled receiving unit 30 is driven in one of approximately horizontal directions by the scanning unit 40. A typical movement direction of the coupled receiving unit 30 in the present invention is an approximately horizontal direction.

The scanning unit 40 is constituted by an XY stage, a ball screw, a stepping motor, and the like, and changes a relative positional relationship between the holding member 22 configured to hold the object 201 and the coupled receiving unit 30 including the receiving portion 33. Accordingly, effects including being able to measure an object over a wide range and improving reconstruction accuracy or an SN ratio by receiving acoustic waves from various directions can be obtained. The scanning unit 40 performs scanning in accordance with control by a scan control unit (not shown). The scan control unit acquires a position of the scanning unit and sends a control signal to the scanning unit in accordance with a program determined in advance or an instruction from the user via input means. Accordingly, the coupled receiving unit 30 moves along a trajectory such as a raster scan or a spiral scan. The scan control unit may be constituted by an independent control circuit or the information processing unit (to be described later) may double as the supply control unit.

Other Components

The acoustic wave apparatus 100 may be provided with the storage unit (not shown) which stores a digital electrical signal output from the signal processing unit and the information processing unit (not shown) which applies an image reconstruction process to the digital electrical signal and generates image data of the object. The information processing unit is typically constituted by a processor such as a CPU or a GPU, a circuit such as an FPGA or an ASIC, a storage unit such as a flash memory or an HDD, communicating means, input/output means, and the like. Any method such as phasing addition, Fourier transform, and back-projection can be used in the image reconstruction process.

The acoustic wave apparatus 100 may be provided with a display unit (not shown) which displays image data output from the information processing unit. Any display apparatus such as a liquid crystal display or an organic EL display can be used as the display unit.

When the acoustic wave apparatus 100 is a photoacoustic imaging apparatus, the apparatus is provided with a light source and an optical system. The light source irradiates light with a wavelength which is absorbed by a specific component (for example, hemoglobin) among components constituting a living organism. The wavelength of light typically ranges from at least 500 nm to not more than 1200 nm. A laser capable of irradiating large-output pulsed light is favorable as the light source. However, a flash lamp or a light-emitting diode may be used in place of a laser. As the laser, various lasers such as a solid-state laser, a gas laser, a dye laser, and a semiconductor laser can be used. In addition, using a wavelength-variable laser or a plurality of lasers with irradiation light of mutually-different wavelengths enables a concentration of a substance such as oxygen saturation to be measured. Light generated from the light source is irradiated to the object 201 from a light irradiating portion 170 via an optical system such as a lens, a mirror, an optical fiber, or a diffuser plate.

A light irradiating portion 170 which emits light guided from a light source 160 may be provided in, for example, a bottom part of a hemispherical portion constituting the receiving portion. In this case, a material that transmits light from the light source is used as the holding member 22. According to this arrangement, the light irradiating portion 170 and the receiving portion can be installed at approximately equal positions as viewed from the object 201. As a result, even when a position at which a photoacoustic wave is generated changes due to scanning, an acoustic wave with high intensity can be received in an efficient manner. However, a light irradiation direction is not limited thereto and, for example, light may be irradiated via the holding member 22 from a side surface of the object 201. In other words, the light irradiating portion 170 is arranged so as to irradiate the object 201 with light via the acoustic matching liquid 32. In addition, the light irradiating portion 170 can be described as being optically coupled to the light source 160 via an optical fiber 180.

On the other hand, when the acoustic wave apparatus 100 is an apparatus that uses ultrasonic echo, an ultrasonic wave may be transmitted from the receiving portion 33 to the object 201. Alternatively, an ultrasonic wave transmitting unit may be provided separately from the receiving portion 33.

Bank Portion

The bank portion 34 that is a feature of the present invention will now be described. The bank portion 34 according to the present embodiment is a portion that protrudes from an upper part of the liquid vessel wall 31w toward a lower surface of the supporting top plate 24 constituting the supporting base 20. In other words, the bank portion 34 according to the present embodiment is located at a upper portion of the liquid vessel 31 configured to reduce an amount of the matching liquid 32 over flowed from the liquid vessel 31 due to a changing of a liquid level of the matching liquid 32. The bank portion 34 is continuously provided in a circumferential direction, excluding a communicating portion to be described later. The bank portion is include in the liquid vessel, and is configured to reduce an amount of the matching liquid over flowed from the liquid vessel due to a changing of a wavefront of the matching liquid. In addition, an upper end of the bank portion 34 is included in a virtual plane in accordance with the supporting top plate 24 with a flat plate shape. A shape of the bank portion 34 when viewed from above approximately coincides with an outer circumferential shape of the liquid vessel 31 when viewed from above. For example, when the liquid vessel 31 has a circular shape in a plan view, the bank portion 34 also has a circular shape in a plan view. By being continuously provided in the circumferential direction, the bank portion is capable of reducing an amount of the acoustic matching liquid 32 that overflows to the outside of the liquid vessel 31 when the acoustic matching liquid 32 is ruffled due to scanning of the liquid vessel 31. In other words, the liquid level of the matching liquid 32 might be changed during a scanning step in which the liquid vessel 31 is moved by the scanning unit 40.

At least a contact portion 34c which is a portion that comes into contact with the supporting top plate 24 in the bank portion 34 is configured so as to be slidable and stretchable. In order to make the contact portion 34c slidable and stretchable, the bank portion 34 (or the contact portion 34c in the bank portion 34) may be constituted by an elastic member such as sponge or rubber. Alternatively, a root of the bank portion 34 may be elastically supported on an upper end portion of the liquid vessel wall 31w. Therefore, even when the coupled receiving unit 30 is driven by the scanning unit 40, the sponge or the rubber slides and stretches and a contact state between the bank portion 34 and the lower surface of the supporting base 20 is maintained. The entire bank portion 34 may have slidability and stretchability.

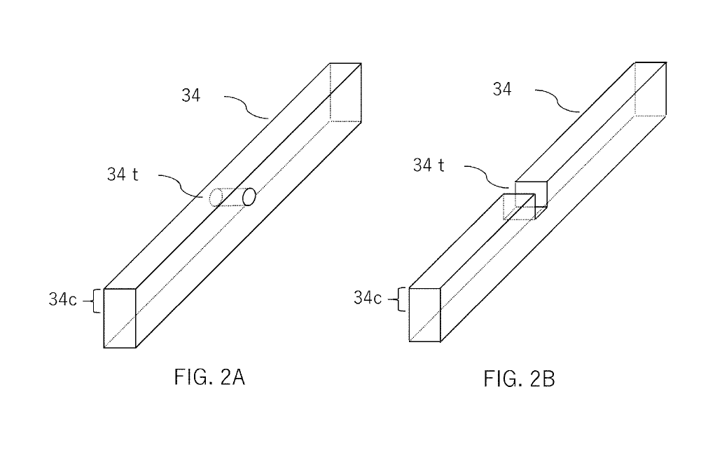

FIGS. 2A and 2B are partial enlarged views of the bank portion 34. As shown, in order to cause the acoustic matching liquid 32 to overflow, a communicating portion 34t is provided in a part of the bank portion 34 that is continuously provided in the circumferential direction. The communicating portion 34t is provided in order to secure a path that enables a fluid to pass between the inside and the outside of the liquid vessel 31. For example, the communicating portion 34t has a hole shape such as that shown in FIG. 2A or a groove shape such as that shown in FIG. 2B. The presence of the communicating portion 34t enables the acoustic matching liquid 32 to be released to the outside of the liquid vessel 31 when an amount of the acoustic matching liquid 32 stored inside the liquid vessel 31 increases.

The contact portion 34c of the bank portion 34 maintaining a contact state with the lower surface of the supporting base 20 as described above produces an effect of preventing outflow of the acoustic matching liquid 32. On the other hand, according to studies carried out by the applicants, it was found that, when the acoustic matching liquid 32 is excessively supplied in excess of a capacity of the liquid vessel 31 for some reason, there is a risk that the overflowed acoustic matching liquid 32 may leak onto the upper surface of the supporting base 20 from a gap between the holding member 22 and the securing portion 23. As a result, there is a possibility that the leakage of liquid may impart a sense of discomfort to the examinee 200.

However, providing the communicating portion 34t which allows liquid to pass through as in the present embodiment, even when a supply amount of the acoustic matching liquid 32 exceeds the capacity of the liquid vessel 31, the overflowed acoustic matching liquid 32 flows out from the opening of the communicating portion 34t instead of the gap between the securing portion 23 and the holding member 22. As a result, liquid leakage no longer imparts a sense of discomfort to the examinee.

Modifications

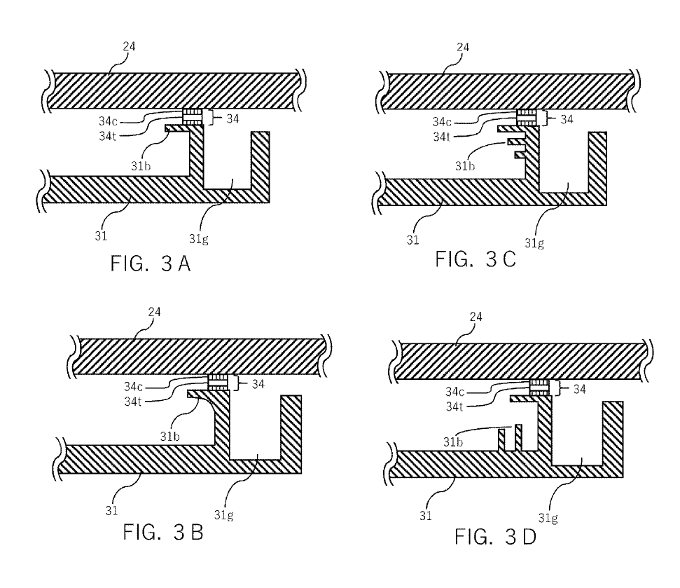

FIGS. 3A to 3D show enlarged sectional views for explaining a modification related to a structure of a periphery of the bank portion 34. The fact that providing the bank portion 34 with the communicating portion 34t prevents the acoustic matching liquid 32 during an overflow from leaking onto an upper part of the supporting top plate and prevents the examinee 200 from getting wet is similar to the embodiment described above. In modifications shown in FIGS. 3A to 3D, a liquid vessel outer moat portion 31g is further provided outside the liquid vessel 31. The liquid vessel outer moat portion 31g is a gutter-shaped member provided so as to surround an outer upper part of the liquid vessel 31. Due to the presence of the liquid vessel outer moat portion 31g, the acoustic matching liquid 32 having escaped from the communicating portion 34t is prevented from scattering inside the apparatus. In this case, the recovery port 37 may be provided in the liquid vessel outer moat portion 31g.

In addition, the surface of the acoustic matching liquid 32 ruffles when the coupled receiving unit 30 is being driven by the scanning unit 40. In consideration thereof, in the modification shown in FIG. 3A, a wave reducing portion 31b is provided near the liquid vessel wall 31w for the purposes of suppressing the ruffles and preventing the ruffled acoustic matching liquid 32 from adhering to the bottom surface of the supporting top plate 24.

FIGS. 3B to 3D respectively show modifications of the wave reducing portion 31b.

With respect to “horizontal” in the acoustic wave apparatus 100 according to the present invention, there is a tolerance of effective inclination. An upper limit and a lower limit of tan θ which corresponds to an angle of inclination are within ±0.5 mm/m with respect to complete horizontal that is perpendicular to a vertical direction. The upper limit and the lower limit of tan θ which corresponds to the angle of inclination is favorably restricted to within ±0.1 mm/m and more favorably restricted to within ±0.04 mm/m.

### Second Embodiment

The present embodiment is featured by the structure of the bank portion 34 and the number and arrangement in which the communicating portion 34t is provided. Since a configuration of the present embodiment is otherwise similar to that shown in FIG. 1, a description thereof will be omitted.

According to studies carried out by the applicants, causes of an overflow of the acoustic matching liquid 32 include a case where the liquid surface of the acoustic matching liquid 32 ruffles significantly in addition to a case where a problem occurs in control by the supply control unit and the acoustic matching liquid 32 is supplied in an amount exceeding the capacity of the liquid vessel 31. In addition, as a result, there is a possibility that an amount of liquid spilling toward the outside of the coupled receiving unit 30 problematically increases.

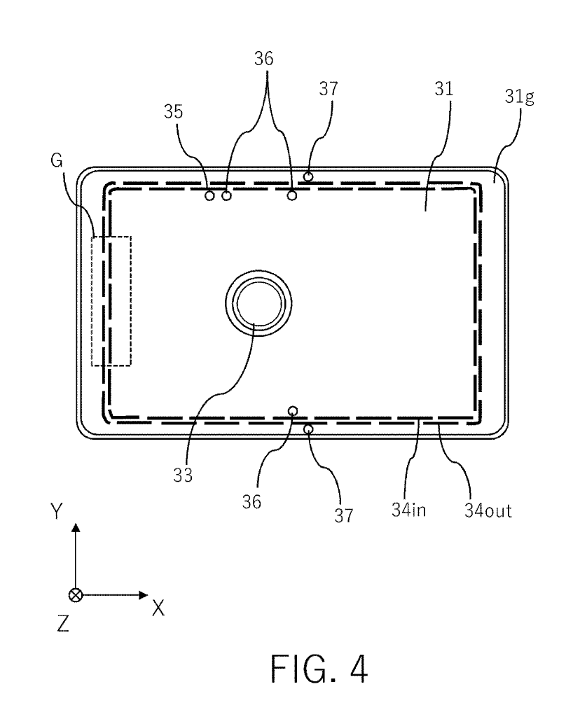

FIG. 4 is a plan view of the coupled receiving unit 30 of the acoustic wave apparatus 100 according to the present embodiment. In the present embodiment, the bank portion 34 is doubly provided along an inner circumferential side (34in) and an outer circumferential side (34out). In other words, in the present embodiment, two rows of bank portions (34in and 34out) are provided in a radial direction. Moreover, the radial direction corresponds to a direction that intersects with the circumferential direction in which the bank portion 34 extends and is defined as one of a direction from an inner side toward an outer side of the bank portion 34 and a direction from the outer side toward the inner side of the bank portion 34. In the specification of the present application, the radial direction is defined as the direction from the inner side toward the outer side of the bank portion 34 so as to correspond to a discharge direction of the acoustic matching liquid that is discharged from the bank portion 34.

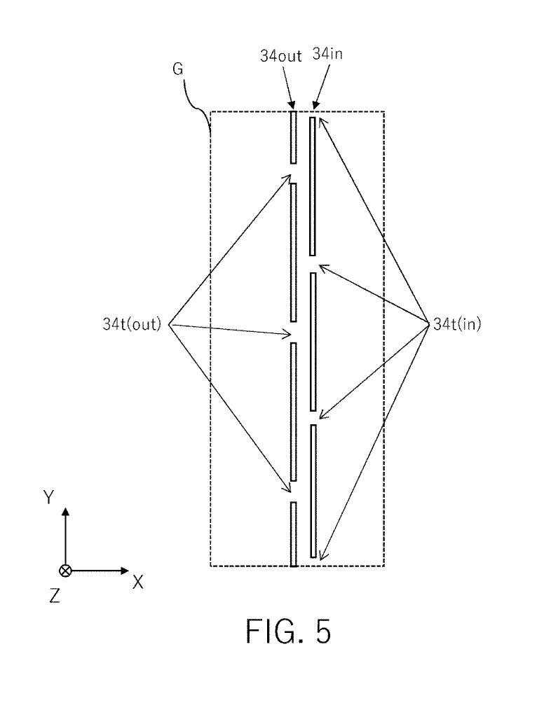

FIG. 5 is a partial enlarged view that enlarges the bank portion 34 in a region G enclosed by a dashed line in FIG. 4. An inner circumferential-side bank portion 34in has a communicating portion 34t (in) discretely provided in plurality in the circumferential direction. In a similar manner, an outer circumferential-side bank portion 34out has a communicating portion 34t (out) discretely provided in plurality in the circumferential direction. Although the communicating portion 34t in FIG. 5 has a groove-like shape, the communicating portion 34t may be shaped like a through-hole instead. In addition, communicating portions with different shapes may be used between the inner-side communicating portion and the outer-side communicating portion. Using double bank portions in this manner enables an amount of liquid spillage to the outside to be suppressed even when the surface of the acoustic matching liquid 32 is ruffled by scanning.

In addition, as shown in FIG. 5, it is also favorable to arrange the inner circumferential-side communicating portion 34t (in) and the outer circumferential-side communicating portion 34t (out) at positions that do not overlap with each other in the circumferential direction. According to such a configuration, the amount of liquid spillage to the outside can be further suppressed. Moreover, the bank portion 34 may also be provided in triplicate or more instead of in double.

### Third Embodiment

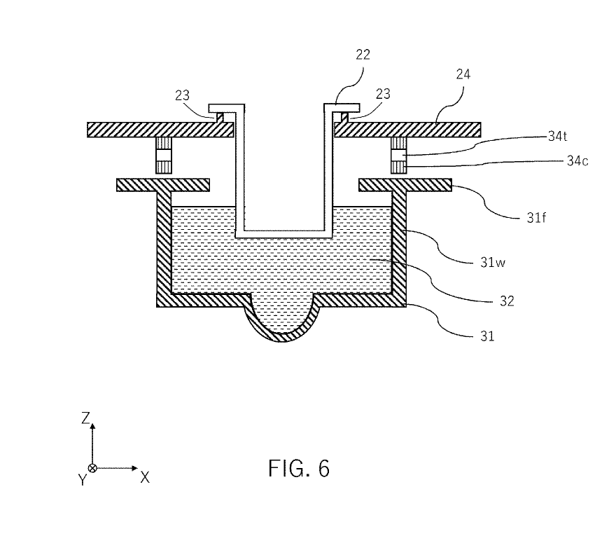

The present embodiment is featured by a location at which the bank portion 34 is arranged. Since a configuration of the present embodiment is otherwise similar to the respective embodiments described above, a description thereof will be omitted. FIG. 6 is a sectional view of a range centered on the liquid vessel 31 and the supporting base 20 in the acoustic wave apparatus 100 according to the present embodiment.

The bank portion 34 according to the present embodiment is formed fixed to the lower surface of the supporting top plate 24 constituting the supporting base 20 and protrudes toward the liquid vessel 31. In the bank portion 34, at least a lower part (a side in contact with the liquid vessel that is another member) is formed of a slidable and stretchable member and constitutes the contact portion 34c. In addition, the bank portion 34 is provided with the communicating portion 34t.

Furthermore, a bank portion receiving portion 31f is provided at a position opposing the bank portion 34 in the upper part of the liquid vessel wall 31w. Accordingly, even when the liquid vessel 31 moves in an XY direction due to scan control, liquid is maintained in a space enclosed by the bank portion 34 and the liquid vessel 31 as long as the bank portion receiving portion 31f and the contact portion 34c at a tip of the bank portion 34 are in contact with each other. The bank portion receiving portion 31f is made in a shape that does not physically interfere with the holding member 22 even when the liquid vessel 31 is scanned. For example, when the liquid vessel 31 is approximately circular in a plan view, the bank portion receiving portion 31f favorably has a donut shape. Moreover, a small bank portion that protrudes upward may be further provided on an inner circumferential side and/or an outer circumferential side of the bank portion receiving portion 31f to suppress outflow of liquid.

As shown in the present embodiment, the bank portion 34 need only have a shape that protrudes from one of the liquid vessel and the supporting base toward the other. Even with the configuration according to the present embodiment, since the acoustic matching liquid 32 flows out from the opening of the communicating portion 34t instead of the gap between the securing portion 23 and the holding member 22, the examinee can be prevented from experiencing discomfort due to leakage. Moreover, the bank portion 34 may be doubly provided in the radial direction in a similar manner to the second embodiment. In doing go, the communicating portions 34t are favorably arranged so as not to overlap with each other between the inner-side bank portion and the outer-side bank portion. Even in the configuration of the present embodiment, favorably, the bank portion 34 (or at least the contact portion 34c) is constituted by an elastic member or the bank portion 34 is elastically supported on the supporting base 20.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-150346, filed on Aug. 3, 2017, which is hereby incorporated by reference herein in its entirety.

What is claimed is:

1. An acoustic wave apparatus, comprising:
a supporting base provided with an insertion opening into which an object is to be inserted;
a liquid vessel arranged below the insertion opening configured to store acoustic matching liquid; and
a receiving portion configured to receive an acoustic wave generated at the object and propagated via the acoustic matching liquid from;
a bank portion protruding from the liquid vessel toward the supporting base, wherein
the liquid vessel includes a bank portion at upper side configured to reduce an amount of the matching liquid over flowed from the liquid vessel due to a changing of a wavefront of the matching liquid,
in a part of a circumferential direction of the liquid vessel, the bank portion is provided with a communicating portion through which the acoustic matching liquid can pass.

2. The acoustic wave apparatus according to claim 1, wherein
the bank portion is continuously provided in the circumferential direction.

3. The acoustic wave apparatus according to claim 1, wherein
the communicating portion is discretely provided in plurality in the circumferential direction.

4. The acoustic wave apparatus according to claim 1, wherein
the bank portion is provided in plurality in a radial direction of the liquid vessel.

5. The acoustic wave apparatus according to claim 4, wherein
the communicating portion is discretely provided in plurality in the circumferential direction in the bank portions provided in plurality in the radial direction, and
the communicating portion provided in the bank portion on an inner circumferential side and the communicating portion provided in the bank portion on an outer circumferential side are arranged so as not to overlap with each other in the circumferential direction.

6. The acoustic wave apparatus according to claim 1, wherein
the receiving portion is provided in the liquid vessel.

7. The acoustic wave apparatus according to claim 1, further comprising a scanning unit configured to move the liquid vessel in an approximately horizontal direction.

8. The acoustic wave apparatus according to claim 1, wherein
the liquid vessel includes a supply port configured to supply the acoustic matching liquid thereinto.

9. The acoustic wave apparatus according to claim 1, wherein
the bank portion is formed of an elastic member or the bank portion is elastically supported by one of the liquid vessel and the supporting base.

10. The acoustic wave apparatus according to claim 1, wherein
the bank portion is fixed to one of the liquid vessel and the supporting base so as to be slidable to the other of the liquid vessel and the supporting base.

11. The acoustic wave apparatus according to claim 1, wherein
the liquid vessel forms a container including a liquid vessel wall and a bottom portion configured to store the acoustic matching liquid.

12. The acoustic wave apparatus according to claim 11, wherein
the bank portion protrudes from the liquid vessel wall toward a lower surface of the supporting base.

13. The acoustic wave apparatus according to claim 11, wherein
the bank portion protrudes from a lower surface of the supporting base toward the liquid vessel wall, and
the liquid vessel is provided with a bank portion receiving portion that opposes the bank portion in an upper part of the liquid vessel wall.

14. The acoustic wave apparatus according to claim 1, wherein
the supporting base includes a securing portion to which a holding member for holding the object is configured to be secured at a position which overlaps with the insertion opening.

15. The acoustic wave apparatus according to claim 1, further comprising a light irradiating portion configured to irradiate the object with light via the acoustic matching liquid.

16. The acoustic wave apparatus according to claim 15, wherein
the light irradiating portion is optically coupled to a light source.

17. The acoustic wave apparatus according to claim 16, further comprising the light source configured to generate the light.
